(19) United States
(12) Patent Application Publication
(10) Pub. No.: US 2023/0056811 A1
(43) Pub. Date: Feb. 23, 2023

(54) MUCOADHESIVE SOLID OR SEMISOLID OCULAR DELIVERY SYSTEMS BASED ON PREACTIVATED THIOMERS

(71) Applicant: BIOADHESIVE OPHTHALMICS, Orsay (FR)

(72) Inventor: Jean GARREC, Paris (FR)

(73) Assignee: BIOADHESIVE OPHTHALMICS, Orsay (FR)

(21) Appl. No.: 17/796,183

(22) PCT Filed: Feb. 5, 2021
(86) PCT No.: PCT/EP2021/052796

§ 371 Date: Jul. 28, 2022

(30) Foreign Application Priority Data
Feb. 7, 2020 (EP) 20305118.0

(51) Int. Cl. A61K 9/00; A61K 47/32; A61P 27/06; A61P 27/02; A61K 31/5575; A61K 31/573; A61K 47/36; D01D 5/00; D01F 1/10

(52) U.S. Cl. CPC A61K 9/0051; A61K 47/32; A61P 27/06; A61P 27/02; A61K 31/5575; A61K 31/573; A61K 47/36; D01D 5/0007; D01F 1/10; D10B 2509/00

(57) ABSTRACT

A mucoadhesive solid or semisolid ocular delivery systems that includes a matrix of preactivated thiomers, preferably under the form of ocular inserts or ocular films. The ocular delivery systems are useful for ocular drug delivery for the treatment of ocular diseases. The delivery systems of the invention can also be used for alleviating ocular conditions such as dry eye syndrome.

### FIELD OF INVENTION

The present invention relates to the field of ophthalmic formulations and provides mucoadhesive solid or semisolid ocular delivery systems. The delivery systems of the invention are based on a matrix of preactivated thiomers and are preferably under the form of ocular inserts or ocular films. The delivery systems of the invention are useful for ocular drug delivery for the treatment of ocular diseases such as, but not limited to, dry eye or glaucoma. The delivery systems of the invention can also be used for alleviating ocular conditions.

### BACKGROUND OF INVENTION

There are numerous ocular diseases and ocular conditions that necessitate the administration of an active substance directly at the eye level. One of the major problems of topical ocular administration is to obtain and maintain a sufficient amount of active substance at the site of action for a prolonged period of time, and to deliver a precise dose of active substance. Delivery systems enabling a controlled sustained release of the active substance overtime are also needed.

Most of the ophthalmic drugs are administered under the form of aqueous eye drops. Aqueous eye drops are easy to use and well accepted by patients, but present the drawback to have a rapid draining after instillation, resulting in poor bioavailability. Moreover, many ophthalmic drugs are hydrophobic molecules which have poor solubility in water. Therefore, many eye drops formulations are drug suspensions, which exacerbates the bioavailability problem since the drug needs first to solubilize in the eye before being absorbed.

Various alternatives were developed in order to optimize ocular medication such as for example using reverse emulsions, in situ gelling polymers, microspheres, nanoparticles, liposomes or ocular inserts.

Ocular inserts are solid or semisolid ocular delivery devices to be placed in the conjunctival cul-de-sac of the eye. Ocular inserts offer an interesting alternative to eye drops since they ensure a longer pre-corneal residence time and reduce the amount of systemic absorption. Nevertheless, ocular inserts most often have poor patient acceptance since they lead to a sensation of foreign body in the eye. Further, in the event that the insert moves around the eye, it may also interfere with vision and cause irritation.

Efforts are thus undertaken to provide mucoadhesive ocular inserts which are well tolerated by patients and remain simple to produce.

When an ocular delivery system is placed at the surface of the eye, it is first in contact with the tear film, which is formed of three layers: lipid layer, aqueous layer and mucin layer. The mucins present under the tear film can thus be targeted in order to obtain the adhesion of the ocular delivery system to the eye surface. Several polymers were already tested for their mucoadhesive properties, such as thiomers.

Thiomers, also referred to as “thiolated polymers”, are polymers having side chains bearing free thiol moieties (Bernkop-Schnürch A. et al., Pharm. Res., 1999, 16, 876-881; U.S. Pat. No. 7,354,600). The polymeric backbone of thiomers usually consists of biodegradable polymers, such as for example chitosan, hyaluronic acid, gelatin, polyacrylates, cyclodextrins or silicones. The thiolation of such polymeric backbones may be performed for example by coupling cysteine moieties. Thiomers are capable of forming covalent bonds, namely disulfide bonds, with cysteine-rich subdomains of mucins covering mucosal membranes. Such covalent bonds are strong and thus enable to ensure an efficient mucoadhesion of dosage forms comprising thiomers for a prolonged time.

Hornof et al. tested a mucoadhesive ocular insert based on thiolated poly(acrylic acid), for the controlled release of ophthalmic drugs (Hornof et al., J. Controlled Release, 2003, 419-428). The dry ocular insert is placed in the conjunctival cul-de-sac of the eye and hydrates in situ to form a hydrogel that presents a good mucoadhesion. The hydrogel form does not lead to a foreign body sensation, contrary to previous ocular inserts and mucoadhesion allows the insert to stay in place. Nevertheless, such thiomer ocular inserts must remain stored at a non-physiological pH (for the thiomer ocular insert of Hornof et al., at pH 5) in order to avoid the oxidation of the thiol groups of the thiomer and maintain them under reduced form. This is essential in order to keep a sufficient amount of free thiol groups available for interaction with mucins. Therefore, the main drawback of thiomer ocular inserts is that they provoke irritation and pain due to their non-physiological pH. Moreover, such pH may not be suitable to carry some active substances which are not stable under such conditions.

There is thus a need for new solid or semisolid ocular delivery systems (including ocular inserts and ocular films) which have effective mucoadhesive properties, are well tolerated by patients and are suitable for the delivery of a wide range of ophthalmic drugs.

For that purpose, the Applicant herein provides mucoadhesive solid or semisolid ocular delivery systems based on a matrix of preactivated thiomers.

Preactivated thiomers, or S-protected thiomers, are thiomers in which the thiol moieties of the side chains are conjugated in disulfide bonds with mercaptonicotinic acids, mercapto(iso)nicotinamides or mercatopyridoxines (US 2012/0225024). Mucoadhesive properties of such preactivated thiomers was reported, for example with poly(acrylic acid)-cysteine-2-mercaptonicotinic acid (Iqbal J. et al., Biomaterials, 2012, 33, 1528-1535). Nevertheless, to the knowledge of the Applicant, the use of such preactivated thiomers was never reported for the manufacturing of ocular delivery systems, especially ocular inserts or ocular films.

The presence of preactivated thiol groups in the thiomers used in ocular delivery systems of the invention enhances the stability, mucoadhesion, cohesive properties and tolerance of the thiomers, and thus provides ocular delivery systems with expected properties. The delivery systems of the invention especially present the advantage to prolong the residence time of the delivery system at the site of application without causing irritation. The adherence to the treatment by patients is improved when using the delivery system of the invention, compared with the use of eye drops, since it avoids repeated instillations.

When used to deliver ophthalmic drugs, the preactivated thiomer-based ocular delivery systems of the invention improve the therapeutic performance of the drug by increasing its bioavailability. Especially, the ocular delivery systems of the invention enable to increase the residence time of the drug, which in turn enhances the permeation of the drug. It also enables its sustained release overtime and allows the delivery of a more precise dose, compared to what can be achieved when using eye drops.

To be suitable for ocular use, the solid or semisolid delivery systems of the present invention should encounter several specifications, such as having a shape and a size adapted to ocular placement, and enabling a suitable hydration with a controlled swelling.

### SUMMARY

This invention thus relates to a mucoadhesive solid or semisolid ocular delivery system comprising a preactivated thiomer matrix, wherein the matrix comprises at least one preactivated thiomer selected from polymeric compounds bearing 2-mercaptonicotinic acid, 6-mercaptonicotinic acid, 2-mercaptonicotinamide, 2-mercaptoisonicotinamide, 6-mercaptonicotinamide, 6-mercaptoisonicotinamide, 6,6′-dithionicotinamide or 6-mercaptopyridoxine side chains covalently bonded through disulfide bonds to a thiolated polymer backbone.

In one embodiment, the ocular delivery system is an ocular insert or an ocular film.

In one embodiment, the polymer backbone is selected from a (crosslinked) homo- or co-polymer consisting of (meth)acrylic acid, (meth)acrylic acid esters, (meth)acrylamides, vinylpyrrolidone, vinylalcohol, vinylimidazole, vinylcaprolactam, divinyl glycol, polycarbophil, carbomer, allylamine, (trimethylated) chitosans, hyaluronic acid, pectins, alginates, (crosslinked) polyallylamines, polylysine, polyornithine, polyaminoamides, methylcellulose, ethylcellulose, hydroxyethylcellulose, hydroxypropylcellulose, hydroxypropylmethylcellulose, and sodium carboxymethylcellulose, optionally exhibiting free thiol groups as side chains.

In one embodiment, the side chains of the polymeric compound are selected from: S-(2- or 6-mercapto(iso)nicotinamide)- or S-(6-mercaptopyridoxine)-cysteine-disulfides, S-(2- or 6-mercapto(iso)nicotinamide)- or S-(6-mercaptopyridoxine)-homocysteine-disulfides, S-(2- or 6-mercapto(iso)nicotinamide)- or S-(6-mercaptopyridoxine)-cysteamine-disulfides, S-(2- or 6-mercapto(iso)nicotinamide)- or S-(6-mercaptopyridoxine)-N-acetylcysteine-disulfides, S-(2- or 6-mercapto(iso)nicotinamide)- or S-(6-mercaptopyridoxine)-thioglycolic acid-disulfides, S-(2- or 6-mercapto(iso)nicotinamide)- or S-(6-mercaptopyridoxine)-3-thiopropionic acid-disulfides, S-(2- or 6-mercapto(iso)nicotinamide)- or S-(6-mercaptopyridoxine)-4-thiobutanoic acid-disulfides, S-(2- or 6-mercapto(iso)nicotinamide)- or

S-(6-mercaptopyridoxine)-mercaptobenzoic acid-disulfides, S-(2- or 6-mercapto(iso)nicotinamide)- or S-(6-mercaptopyridoxine)-mercaptonicotinic acid-disulfides, S-(2- or 6-mercapto(iso)nicotinamide)- or S-(6-mercaptopyridoxine)-glutathione-disulfides, S-(2- or 6-mercapto(iso)nicotinamide)- or S-(6-mercaptopyridoxine)-thioethylamidine-disulfides, S-(2- or 6-mercapto(iso)nicotinamide)- or S-(6-mercaptopyridoxine)-4-thiobutylamidine-disulfides, and S-(2- or 6-mercapto(iso)nicotinamide)- or S-(6-mercaptopyridoxine)-mercaptoaniline-disulfides; said side chains being attached to the polymer backbone via amide, amidine or ester bonds.

In one embodiment, the preactivated thiomer forming the matrix is under the form of nanofibers; preferably nanofibers obtained by electrospinning.

In one embodiment, the ocular delivery system of the invention further comprises one or more pharmaceutically active substance; preferably the pharmaceutically active substance is selected from intraocular pressure (IOP) lowering agents such as prostaglandin analogs, cholinomimetics, beta blockers, alpha adrenergic agonists, carbonic anhydrase inhibitors, Rho kinase inhibitors, NO donor agents and combinations thereof; anti-inflammatories such as corticosteroid anti-inflammatory drugs and nonsteroidal anti-inflammatory drugs; anti-infectives such as macrolides, aminosides, rifamycin, antiviral drugs and antifungal medication; anti-allergic agents such as H1-antihistamines, and mast cell stabilizers; and dry eye treatment agents.

In one embodiment, the ocular delivery system of the invention further comprises one or more pharmaceutically acceptable excipient selected from: thickening agents, gelling agents, plasticizers, solubilization agents, stabilizing agents, permeation enhancers, diluents, binding agents, disintegrants, channeling agents, glidants and buffering agents. In one embodiment, the thickening and gelling agents are selected from high molecular weight crosslinked polyacrylic acid polymers, polyvinyl alcohol, polyvinylpyrrolidone, cellulose derivatives, polyethylene glycol, and hyaluronic acid; plasticizer is glycerol; solubilization and stabilizing agents are selected from cyclodextrins; the permeation enhancer is glutathione in its reduced form; the diluents are selected from sugar alcohols; the binding agents are selected from saccharides and their derivatives, disaccharides, polysaccharides and their derivatives, cellulose, modified cellulose, sugar alcohols and synthetic polymers; disintegrants are selected from crosslinked polymers and modified starch; glidants are selected from magnesium stearate, dibasic calcium phosphate, starch, microcrystalline cellulose and colloidal silicon dioxide; and channeling agents are selected from sodium chloride and polyethylene glycol.

In one embodiment, the ocular delivery system of the invention comprises one or more pharmaceutically acceptable excipient selected from high molecular weight crosslinked polyacrylic acid polymers, polyvinyl alcohol, polyvinylpyrrolidone, cellulose, microcrystalline cellulose, hydroxypropyl cellulose, hydroxypropyl methylcellulose, carboxymethyl cellulose, polyethylene glycol, hyaluronic acid, glycerol, cyclodextrins, glutathione in its reduced form, sorbitol, xylitol, mannitol, saccharides and their derivatives, sucrose, lactose, polysaccharides and their derivatives, starches, sodium starch glycolate, magnesium stearate, dibasic calcium phosphate, colloidal silicon dioxide and sodium chloride.

The present invention also provides a mucoadhesive solid or semisolid ocular delivery system for use in the treatment and/or prevention of an ocular disease or ocular condition. In one embodiment, the ocular disease or ocular condition is selected from open angle glaucoma, macular edema, uveitis, dry eye disease, conjunctivitis, keratitis, blepharitis, endophthalmitis, trachoma, post-surgical and seasonal allergies.

The invention further relates to the use of a preactivated thiomer for the manufacturing of a mucoadhesive solid or semisolid ocular delivery system, preferably an ocular insert or an ocular film, wherein the preactivated thiomer is selected from polymeric compounds bearing 2-mercaptonicotinic acid, 6-mercaptonicotinic acid, 2-mercaptonicotinamide, 2-mercaptoisonicotinamide, 6-mercaptonicotinamide, 6-mercaptoisonicotinamide, 6,6′-dithionicotinamide or 6-mercaptopyridoxine side chains covalently bonded through disulfide bonds to a thiolated polymer backbone.

### Definitions

In the present invention, the following terms have the following meanings:

The term “administration”, or a variant thereof (e.g. “administering”), means providing the active substance, alone or as part of a pharmaceutically acceptable formulation, to the patient in whom/which the condition, symptom, or disease is to be treated or prevented.

The term “carbomer” refers to synthetic high-molecular-weight polyacrylic acids cross-linked with allyl sucrose or allyl pentaerythritol. Examples of carbomers include Carbopol 971 and 974 which are polyacrylic acids cross-linked with allyl pentaerythritol and polymerized in ethyl acetate.

The term “electrospinning” refers to a process that generates a network of tridimensional polymer nanofibers. Electrospinning uses an electrical charge to draw very fine fibers from a liquid. Methods to perform electrospinning are known by skilled artisan.

The term “human” refers to a subject of both genders and at any stage of development (i.e. neonate, infant, juvenile, adolescent, adult).

The term “mucoadhesive” refers to the attractive forces between a substance or material and mucus or mucosal membrane. In the context of the present invention, a “mucoadhesive ocular delivery system” is an ocular delivery system that strongly interact with mucus or mucosal membranes. In a preferred embodiment, the ocular delivery system of the invention covalently binds to the mucus or mucosal membrane by the formation of disulfide bounds between the thiomer and the natural mucins present therein. This disulfide bound formation is facilitated by the use of preactivated thiomers.

The term “nanofibers” refers to a fiber having an average diameter of less than 5000 nm.

The term “ocular delivery system” refers to a delivery system which enable to administer an active pharmaceutical ingredient or a substance of interest, such as for example a drug or a lubricating agent, to a subject via an eye or any part thereof. A “solid or semisolid ocular delivery system” refers to solid or semisolid dosage forms including ocular inserts and ocular films. Especially, “semisolid” refers to a dosage form which may be highly viscous, such as an ocular insert under the form of a hydrogel.

The term “ocular film” refers to a solid or semisolid consistency bidimensional film designed to be placed into the conjunctival cul-de-sac or at the conjunctival surface, whose size and shape are especially designed for ophthalmic application. Preferably, ocular films are sterile. The ocular film can be folded to form a tridimensional device, what can be useful for example to facilitate the placement of the film on the eye.

The term “ocular insert” refers to a solid or semisolid consistency tridimensional device designed to be placed into the conjunctival cul-de-sac or at the conjunctival surface, whose size and shape are especially designed for ophthalmic application. Preferably, ocular inserts are sterile. Optionally, ocular inserts can be multilayered. Ocular inserts can be under dry or hydrated forms. In the latter case, in the present invention, the ocular insert is under the form of a hydrogel pellet.

The term “ocular disease” refers to any disease that affects any area of the eyeball, including its surface, the anterior and posterior segment of the eye, as well as the eyelids (preferably the interior side of the eyelid). The targeted eye tissue can be, but is not limited to, corneal tissue, conjunctiva, eyelids, trabeculum, iris, ciliary body, uvea, choroid, retina or macula.

The term “ocular condition” refers to any condition that affects any area of the eyeball, as well as the eyelids. Examples of ocular conditions include ocular condition after eye surgery, dry eye symptoms and ocular symptoms due to seasonal allergies.

The term “patient” refers to a mammal, who/which is awaiting the receipt of, or is receiving medical care or is/will be the object of a medical procedure. In one embodiment, the patient is a human. In another embodiment, the patient is an animal.

The expression “pharmaceutically acceptable” refers to the ingredients of a pharmaceutical formulation which are compatible with each other and not deleterious to the subject to which it is administered.

The expression “pharmaceutically acceptable excipient and/or adjuvant” refers to a substance that does not produce an adverse, allergic or other untoward reaction when administered to an animal, preferably a human. It includes any and all inactive substance such as for example solvents, cosolvents, antioxidants, surfactants, stabilizing agents, emulsifying agents, buffering agents, pH modifying agents, preserving agents (or preservating agents), antibacterial and antifungal agents, isotonifiers, granulating agents or binders, lubricants, disintegrants, glidants, diluents or fillers, adsorbents, dispersing agents, suspending agents, coating agents, bulking agents, release agents, absorption delaying agents, sweetening agents, flavoring agents and the like. For human administration, preparations should meet sterility, pyrogenicity, general safety and purity standards as required by regulatory offices, such as, e.g., FDA Office or EMA.

The term “polycarbophil” refers to a synthetic polymer manufactured from the cross-linking of polyacrylic acid with divinyl glycol and a calcium counter-ion.

The term “polymeric compounds” refers to a polymer. In the sense of the present invention, a polymeric compound may comprise a “polymer backbone” with “side chains”.

The terms “prevent”, “preventing” and “prevention”, as used herein, refer to a method of delaying or precluding the onset of a condition or disease and/or its attendant symptoms, barring a patient from acquiring a condition or disease, or reducing a patient's risk of acquiring a condition or disease.

The term “subject” refers to a mammal, including humans and animals. In one embodiment, the subject is diagnosed with a disease. In one embodiment, the subject is a patient, who/which is awaiting the receipt of, or is receiving, medical care or was/is/will be the subject of a medical procedure or is monitored for the development or progression of a disease. In one embodiment, the subject is a patient who is treated and/or monitored for the development or progression of a disease. In one embodiment, the subject is a male.

In another embodiment, the subject is a female. In one embodiment, the subject is an adult. In another embodiment, the subject is a child.

The terms “therapeutically effective amount” or “effective amount” or “therapeutically effective dose” refer to the amount or dose of active substance that is aimed at, without causing significant negative or adverse side effects to the subject, (1) delaying or preventing the onset of a disease in the subject; (2) reducing the severity or incidence of a disease; (3) slowing down or stopping the progression, aggravation, or deterioration of one or more symptoms of a disease affecting the subject; (4) bringing about ameliorations of the symptoms of a disease affecting the subject; or (5) curing a disease affecting the subject. A therapeutically effective amount may be administered prior to the onset of a disease for a prophylactic or preventive action. Alternatively, or additionally, a therapeutically effective amount may be administered after initiation of a disease for a therapeutic action.

The terms “treating” or “treatment” refer to therapeutic treatment; wherein the object is to prevent or slow down the targeted pathologic condition or disease. A subject or mammal is successfully “treated” for a disease or affection or condition if, after receiving the treatment according to the present invention, the subject or mammal shows observable and/or measurable reduction in or absence of one or more of the following: relief to some extent, for one or more of the symptoms associated with the specific disease or condition; and improvement in quality of life issues. The above parameters for assessing successful treatment and improvement in the disease are readily measurable by routine procedures familiar to a physician.

The term “thiomer” or “non-preactivated thiomers” refers to thiolated polymers, i.e. polymers having side chains bearing free thiol moieties. The polymeric backbone can be a biodegradable polymer, such as for example chitosan, hyaluronic acid, gelatin, polyacrylates, cyclodextrins, or silicones. The thiolation of such polymeric backbones may be performed for example by coupling cysteine moieties.

The term “preactivated thiomer” or “S-protected thiomer” refers to thiomers in which the thiol moieties of the side chains are conjugated through disulfide bonds with mercaptonicotinic acids, mercapto(iso)nicotinamides or mercatopyridoxines. Examples of preactivated thiomers are disclosed in US 2012/0225024.

The term “preactivated thiomer matrix” refers to a matrix mainly made of preactivated thiomer.

### DETAILED DESCRIPTION

The present invention thus relates to ocular delivery systems, especially solid or semisolid ocular delivery systems. The ocular delivery systems of the invention present mucoadhesive properties that enable the delivery system to remain on the ocular surface for extended periods of time. The ocular delivery systems of the invention are indeed based on a preactivated thiomer matrix, i.e. a matrix mainly made of one or more preactivated thiomer. Once hydrated, the preactivated thiomer matrix of the ocular delivery system of the invention forms a hydrogel that adheres to the eye.

Therefore, according to one embodiment, the invention provides a mucoadhesive solid or semisolid ocular delivery system comprising a matrix of preactivated thiomer.

In one embodiment, the matrix comprises at least one preactivated thiomer.

According to one embodiment, preactivated thiomers are selected from polymeric compounds bearing vitamin B3- or vitamin B6-derivatives side chains covalently bonded through disulfide bonds to a thiolated polymer backbone. According to one embodiment, preactivated thiomers are selected from polymeric compounds bearing 2-mercaptonicotinic acid, 6-mercaptonicotinic acid, 2-mercaptonicotinamide, 2-mercaptoisonicotinamide, 6-mercaptonicotinamide, 6-mercaptoisonicotinamide, 6,6′-dithionicotinamide or 6-mercaptopyridoxine side chains covalently bonded through disulfide bonds to a thiolated polymer backbone. According to a preferred embodiment, preactivated thiomers are selected from polymeric compounds bearing 2-mercaptonicotinamide, 2-mercaptoisonicotinamide, 6-mercaptonicotinamide, 6-mercaptoisonicotinamide or 6-mercaptopyridoxine side chains covalently bonded through disulfide bonds to a thiolated polymer backbone.

By “thiolated polymer backbone” it is referred to a polymer bearing free thiol moieties, preferably a polymer backbone bearing side chains comprising free thiol moieties. In preactivated thiomers, mercaptonicotinic acids, mercapto(iso)nicotinamides or mercaptopyridoxines are linked through disulfide bond to part or all of the thiol moieties of a thiolated polymer backbone.

According to one embodiment, the polymer backbone is selected from a (crosslinked) homo- or co-polymer consisting of (meth)acrylic acid, (meth)acrylic acid esters, (meth)acrylamides, vinylpyrrolidone, vinylalcohol, vinylimidazole, vinylcaprolactam, divinyl glycol, polycarbophil, carbomer, allylamine, (trimethylated) chitosans, hyaluronic acid, pectins, alginates, (crosslinked) polyallylamines, polylysine, polyornithine, polyaminoamides and cellulose derivatives (such as methylcellulose, ethylcellulose, hydroxyethylcellulose, hydroxypropylcellulose, hydroxypropylmethylcellulose, and sodium carboxymethylcellulose), optionally exhibiting free thiol groups as side chains. According to one embodiment, the polymer backbone is a biodegradable polymer such as for example alginates, chitosan, hyaluronic acid, gelatin or polyacrylates. According to a specific embodiment, the polymer backbone is selected from polycarbophil, poly(acrylic acid) and carbomers (such as Carbopol 971 NF and Carbopol 974 NF). According to a specific embodiment, the polymer backbone is selected from chitosan, hyaluronic acid and cellulose derivatives (such as methylcellulose, ethylcellulose, hydroxyethylcellulose, hydroxypropylcellulose, hydroxypropylmethylcellulose, and sodium carboxymethylcellulose).

According to one embodiment, the thiolation of the polymer backbone, such as those described above, can be performed by coupling—via amide, amidine or ester bonds—moieties selected from cysteine, homocysteine, cysteamine, N-acetylcysteine, thioglycolic acid, 3-thiopropionic acid, 4-thiobutanoic acid, mercaptobenzoic acid, mercaptonicotinic acid, glutathione, thioethylamidine, 4-thiobutylamidine and mercaptoaniline Preferably, the thiolation of the polymer backbone is performed by coupling cysteine or cysteamine.

According to one embodiment, in the preactivated thiomers used in the delivery system of the invention, the side chains present on the polymer backbone are selected from:
S-(2- or 6-mercaptonicotinic acid)-, S-(2- or 6-mercapto(iso)nicotinamide)-, S-6,6′-dithionicotinamide- or S-(6-mercaptopyridoxine)-cysteine-disulfides,
S-(2- or 6-mercaptonicotinic acid)-, S-(2- or 6-mercapto(iso)nicotinamide)-, S-6,6′-dithionicotinamide- or S-(6-mercaptopyridoxine)-homocysteine-disulfides,
S-(2- or 6-mercaptonicotinic acid)-, S-(2- or 6-mercapto(iso)nicotinamide)-, S-6,6′-dithionicotinamide- or S-(6-mercaptopyridoxine)-cysteamine-disulfides,
S-(2- or 6-mercaptonicotinic acid)-, S-(2- or 6-mercapto(iso)nicotinamide)-, S-6,6′-dithionicotinamide- or S-(6-mercaptopyridoxine)-N-acetylcysteine-disulfides,
S-(2- or 6-mercaptonicotinic acid)-, S-(2- or 6-mercapto(iso)nicotinamide)-, S-6,6′-dithionicotinamide- or S-(6-mercaptopyridoxine)-thioglycolic acid-disulfides,
S-(2- or 6-mercaptonicotinic acid)-, S-(2- or 6-mercapto(iso)nicotinamide)-, S-6,6′-dithionicotinamide- or S-(6-mercaptopyridoxine)-3-thiopropionic acid-disulfides,
S-(2- or 6-mercaptonicotinic acid)-, S-(2- or 6-mercapto(iso)nicotinamide)-, S-6,6′-dithionicotinamide- or S-(6-mercaptopyridoxine)-4-thiobutanoic acid-disulfides,
S-(2- or 6-mercaptonicotinic acid)-, S-(2- or 6-mercapto(iso)nicotinamide)-, S-6,6′-dithionicotinamide- or S-(6-mercaptopyridoxine)-mercaptobenzoic acid-disulfides,
S-(2- or 6-mercaptonicotinic acid)-, S-(2- or 6-mercapto(iso)nicotinamide)-, S-6,6′-dithionicotinamide- or S-(6-mercaptopyridoxine)-mercaptonicotinic acid-disulfides,
S-(2- or 6-mercaptonicotinic acid)-, S-(2- or 6-mercapto(iso)nicotinamide)-, S-6,6′-dithionicotinamide- or S-(6-mercaptopyridoxine)-glutathione-disulfides,
S-(2- or 6-mercaptonicotinic acid)-, S-(2- or 6-mercapto(iso)nicotinamide)-, S-6,6′-dithionicotinamide- or S-(6-mercaptopyridoxine)-thioethylamidine-disulfides,
S-(2- or 6-mercaptonicotinic acid)-, S-(2- or 6-mercapto(iso)nicotinamide)-, S-6,6′-dithionicotinamide- or S-(6-mercaptopyridoxine)-4-thiobutylamidine-disulfides, and
S-(2- or 6-mercaptonicotinic acid)-, S-(2- or 6-mercapto(iso)nicotinamide)-, S-6,6′-dithionicotinamide- or S-(6-mercaptopyridoxine)-mercaptoaniline-disulfides;
said side chains being attached to the polymer backbone via amide, amidine or ester bonds.

It is herein defined that S-(2- or 6-mercaptonicotinic acid)- stands for S-(2-mercaptonicotinic acid)- or S-(6-mercaptonicotinic acid)-; and that S-(2- or 6-mercapto(iso)nicotinamide)- stands for S-(2-mercaptonicotinamide)-, S-(2-mercaptoisonicotinamide)-, S-(6-mercaptonicotinamide)- or S-(6-mercaptoisonicotinamide)-.

According to one embodiment, preactivated thiomers used in the delivery system of the invention are those disclosed in US 2012/0225024, the content of which is herein incorporated by reference.

According to a specific embodiment, the preactivated thiomers used in the invention are selected from polymeric compounds bearing 2-mercaptonicotinic acid, 6-mercaptonicotinic acid, 2-mercaptonicotinamide, 2-mercaptoisonicotinamide, 6-mercaptonicotinamide, 6-mercaptoisonicotinamide, 6,6′-dithionicotinamide or 6-mercaptopyridoxine side chains covalently bonded through disulfide bonds to a thiolated polymer backbone selected from: poly(acrylic acid)-cysteine, polycarbophil-cysteine, carboxymethylcellulose-cysteine, chitosan-cysteine, hydroxypropylcellulose-cysteine, alginate-cysteine, pectin-cysteine, hyaluronic acid-cysteine, (copolymer of acrylic acid and divinyl glycol)-cysteine, poly(acrylic acid)-cysteamine, polycarbophil-cysteamine, carboxymethylcellulose-cysteamine, chitosan-cysteamine, hydroxypropylcellulose-cysteamine, alginate-cysteamine, pectin-cysteamine, hyaluronic acid-cysteamine, (copolymer of acrylic acid and divinyl glycol)-cysteamine.

According to a specific embodiment, the preactivated thiomer used in the delivery system of the invention is selected from poly(acrylic acid)-cycteine-2-mercaptonicotinic acid and chitosan-cycteine-6-mercaptonicotinic acid. According to a specific embodiment, the preactivated thiomer is poly(acrylic acid)-cycteine-2-mercaptonicotinic acid. According to another specific embodiment, the preactivated thiomer is chitosan-cycteine-6-mercaptonicotinic acid.

According to one embodiment, the mucoadhesive properties of the preactivated thiomers can be modulated depending on the molecular weight of the polymer. For anionic as well as for cationic preactivated thiomers, very efficient mucoadhesive properties can be achieved with medium molecular mass, preferably for molecular mass ranging from 100 kDa to 1000 kDa, preferably from 300 kDa to 700 kDa.

The synthesis of preactivated thiomers can be performed by reaction of a thiolated polymer backbone with 2-mercaptonicotinic acid, 6-mercaptonicotinic acid, 2-mercaptonicotinamide, 2-mercaptoisonicotinamide, 6-mercaptonicotinamide, 6-mercaptoisonicotinamide, 6,6′-dithionicotinamide or 6-mercaptopyridoxine.

The manufacturing of the preactivated thiomers used in the invention can be performed according to the methods disclosed in US 2012/0225024.

According to one embodiment, a matrix made of a preactivated thiomers nanofibers network could be formed by applying an electrospinning technique to the preactivated thiomers. This presents the advantage to enable to control the rate of release of the active substance present in the delivery system by varying the density of the network of nanofibers constituting the matrix.

According to one embodiment, the ocular delivery system of the invention does not comprise a non-preactivated thiomer. In another embodiment, the ocular delivery system of the invention comprises at least one preactivated thiomer and at least one non-preactivated thiomer. By “non-preactivated thiomer” it is referred to thiomers.

The present invention also relates to the use of a preactivated thiomer for the manufacturing of a mucoadhesive solid or semisolid ocular delivery system, preferably an ocular insert or an ocular film, wherein the preactivated thiomer is selected from polymeric compounds bearing 2-mercaptonicotinic acid, 6-mercaptonicotinic acid, 2-mercaptonicotinamide, 2-mercaptoisonicotinamide, 6-mercaptonicotinamide, 6-mercaptoisonicotinamide, 6,6′-dithionicotinamide or 6-mercaptopyridoxine side chains covalently bonded through disulfide bonds to a thiolated polymer backbone.

In addition to the preactivated thiomer, excipients and adjuvants may be used to formulate the ocular delivery system of the invention. Preferably, excipients and adjuvants are pharmaceutically acceptable excipients and adjuvants. Such suitable excipients and adjuvants will be clear to the skilled person; reference is made to the latest edition of Remington's Pharmaceutical Sciences.

According to one embodiment, the ocular delivery system of the invention further comprises one or more pharmaceutically acceptable excipient selected from: thickening agents, gelling agents, plasticizers, solubilization agents, stabilizing agents, permeation enhancers, diluents, binding agents, disintegrants, glidants, channeling agents and buffering agents. According to one embodiment, the ocular delivery system of the invention comprises one or more pharmaceutically acceptable excipient selected from: thickening agents, gelling agents, plasticizers, solubilization agents, stabilizing agents, permeation enhancers, diluents, binding agents, glidants and buffering agents.

Examples of thickening and gelling agents include high molecular weight crosslinked polyacrylic acid polymers (e.g. Carbopol), polyvinyl alcohol, polyvinylpyrrolidone, cellulose derivatives (e.g. hydroxypropyl methylcellulose (HPMC), carboxymethylcellulose (CMC), hydroxypropylcellulose (HPC)), polyethylene glycol, and hyaluronic acid.

Examples of plasticizers include glycerol.

Examples of solubilization and stabilizing agents, especially for active pharmaceutical ingredients, include cyclodextrins and non-ionic surfactants.

Examples of permeation enhancers include glutathione in its reduced form (GSH; 0.1%-1%).

Examples of diluents include sugar alcohols such as sorbitol, xylitol or mannitol.

Examples of binding agents include saccharides and their derivatives; disaccharides such as sucrose or lactose; polysaccharides and their derivatives such as starches, cellulose or modified cellulose such as microcrystalline cellulose and cellulose ethers such as hydroxypropyl cellulose (HPC); sugar alcohols such as xylitol, sorbitol or mannitol; synthetic polymers such as polyvinylpyrrolidone (PVP) or polyethylene glycol (PEG).

Examples of disintegrants include crosslinked polymers such as polyvinylpyrrolidone or carboxymethyl cellulose, modified starch such as sodium starch glycolate.

Examples of glidants include magnesium stearate, dibasic calcium phosphate, starch, microcrystalline cellulose and colloidal silicon dioxide.

Examples of buffering agents include phosphate-buffered saline solution.

Examples of channeling agents include sodium chloride (NaCl) and polyethylene glycol of molecular mass of 400 to 1500 g/mol.

According to one embodiment, the ocular delivery system of the invention comprises one or more pharmaceutically acceptable excipient selected from high molecular weight crosslinked polyacrylic acid polymers, polyvinyl alcohol, polyvinylpyrrolidone, cellulose, microcrystalline cellulose, hydroxypropyl cellulose, hydroxypropyl methylcellulose, carboxymethyl cellulose, polyethylene glycol, hyaluronic acid, glycerol, cyclodextrins, glutathione in its reduced form, sorbitol, xylitol, mannitol, saccharides and their derivatives, sucrose, lactose, polysaccharides and their derivatives, starches, sodium starch glycolate, magnesium stearate, dibasic calcium phosphate, colloidal silicon dioxide and sodium chloride.

The excipients and adjuvants present in the ocular delivery system of the invention may enable to control its swelling upon hydration. Indeed, it should be avoided that the system gains too much volume upon hydration, otherwise it will no more be compatible with ocular use.

The excipients and adjuvants present in the ocular delivery system of the invention may also enable to control the hardness of the ocular delivery system, especially in the case of systems used under dry form.

The choice of the excipients and adjuvants present in the ocular delivery system of the invention may also enable to control the rate of release of the active substance present in the delivery system.

The ocular delivery system of the invention enables to deliver an active substance, preferably a pharmaceutically active substance, to the eye of a subject in need thereof.

In one embodiment, the ocular delivery system of the invention further comprises at least one pharmaceutically active substance. In one embodiment, the ocular delivery system of the invention further comprises one or more pharmaceutically active substance. The ocular delivery system of the invention can also comprise a combination of pharmaceutically active substances.

In one embodiment, the pharmaceutically active substance is selected from anti-glaucoma drugs (especially intraocular pressure (IOP) lowering agents), anti-inflammatories, anti-infectives, anti-allergic agents and dry eye treatment agents. Preferably, the pharmaceutically active substance is an ophthalmic drug.

In one embodiment, anti-glaucoma drugs include IOP lowering agents. In one embodiment, the IOP lowering agents are those that may be used in the treatment of open angle glaucoma. Examples of IOP lowering agents comprise prostaglandin analogs, cholinomimetics, beta blockers, alpha adrenergic agonists, carbonic anhydrase inhibitors, Rho kinase inhibitors, NO donor agents and combination thereof. In one embodiment, a combination of one or more IOP lowering agents is used, preferably a combination of at least two classes of IOP lowering agents. Examples of prostaglandin analogs include latanoprost, bimatoprost, travoprost, tafluprost and latanoprostene bunod. In a specific embodiment, the active substance is bimatoprost. Examples of cholinomimetic include pilocarpine, echothiophate and carbachol. Examples of beta blockers include timolol and nadolol. Examples of alpha adrenergic agonists include brimonidine and apraclonidine. Examples of carbonic anhydrase inhibitors include dorzolamide, brinzolamide, acetalozamide and methazolamide. Examples of Rho kinase inhibitors include netarsudil. Examples of NO donor agents include latanoprostene bunod.

In one embodiment anti-inflammatories may be use in post-surgical treatment after ocular surgery, or for the treatment of macular edema, uveitis or in dry eye disease. Examples of anti-inflammatories comprise corticosteroids anti-inflammatory drugs (dexamethasone, fluorometholone, rimexolone, fluocinolone, fluticasone, loteprednol) and nonsteroidal anti-inflammatory drugs (bromfenac sesquihydrate, amfenac, nepafenac, aspirin, ibuprofen, ketorolac, tromethamine, diclofenac, flurbiprofen). In a specific embodiment, the active substance is dexamethasone or a salt thereof, such as dexamethasone phosphate sodium.

In one embodiment, anti-infectives may be used in post-surgical treatment after ocular surgery (including, but not limited to, cataract surgery), or for the treatment of conjunctivitis, keratitis, blepharitis, endophthalmitis, trachoma or any other bacterial infection, fungal infection or viral infections. Examples of anti-infective include but not only macrolides, aminosides, rifamycin, antiviral drugs and antifungal medication (moxifloxacin, natamycin, azithromycin, mupirocin, erythromycin, ciprofloxacin, netilmicin, besifloxacin, gatifloxacin, gentamycin sulfate, levofloxacin, ofloxacin, sulfacetamide sodium, tobramycin, bacitracin zinc, Polymyxin B sulfate, neomycin, and neomycin sulfate, acyclovir, valacyclovir, famciclovir, itraconazole, posaconazole, and voriconazole).

In one embodiment, anti-allergic agents may be use in the context of seasonal allergies. Examples of anti-allergic agents include H1-antihistamines and mast cell stabilizers (oxymetazoline hydrochloride, cetirizine hydrochloride).

In one embodiment, dry eye treatment agents include immunosuppressive agents such as ciclosporin or tacrolimus.

According to a further embodiment, the ocular delivery system of the invention also enables to deliver other active substances, including alleviating agents of ocular conditions such as dry eye. Examples of such alleviating agents include lubricating agents such as polyvinyl acid (PVA) or polyvinylpyrrolidone (PVP).

According to one embodiment, the ocular delivery system of the invention comprising an active substance in an amount ranging from 0.01% to 50% in weight of the total weight of the delivery system; preferably from 0.1% w/w to 20% w/w; more preferably from 0.1% w/w to 10% w/w.

The present invention also relates to the use of the mucoadhesive solid or semisolid ocular delivery system of the invention in the treatment and/or prevention of ocular diseases or ocular conditions. Especially the ocular delivery system of the invention is useful to deliver one or more active substance at the eye level of a subject. The targeted eye tissue can be, but is not limited to, corneal tissue, conjunctiva, eyelids, trabeculum, iris, ciliary body, uvea, choroid, retina or macula. The ocular delivery system of the invention is useful for human and veterinary uses.

In one embodiment, the invention provides a mucoadhesive solid or semisolid ocular delivery system of the invention for use the treatment and/or prevention of ocular diseases or ocular conditions.

The invention also relates to the use of a mucoadhesive solid or semisolid ocular delivery system of the invention for the manufacturing of a medicament for the treatment and/or prevention of ocular diseases or ocular conditions.

The invention further relates to a method for treating and/or preventing ocular diseases or ocular conditions in a patient, comprising administering to target site of the eye of the patient in need thereof a mucoadhesive solid or semisolid ocular delivery system according to the invention. The mucoadhesive solid or semisolid ocular delivery system of the invention is preferably placed in the cul-de-sac of the eye of the patient in need thereof.

The ocular delivery system of the invention enables to treat diseases of anterior segment of the eye, as well as diseases of the posterior segment of the eye.

According to one embodiment, the ocular diseases or ocular conditions are selected from open angle glaucoma, macular edema, uveitis, dry eye disease, conjunctivitis, keratitis, blepharitis, endophthalmitis, trachoma, post-surgical and seasonal allergies. In one embodiment, the ocular disease is dry eye. In one embodiment, the ocular disease is open angle glaucoma.

According to one embodiment, the mucoadhesive solid or semisolid ocular delivery system of the invention may also be used for the alleviation of dry eye symptoms. This is especially effective when the ocular delivery system comprises a lubricating agent, as mentioned above.

In one embodiment, the mucoadhesive solid or semisolid ocular delivery system of the invention is in unit dosage form.

The ocular delivery system of the invention can be used under dry or hydrated form. In one embodiment, the ocular delivery system of the invention is under dry form, i.e. it contains a limited, if any, amount of water. In such case, once placed into the conjunctival cul-de-sac or at the conjunctival surface, the delivery system of the invention hydrates in situ and the hydrated preactivated thiomer matrix forms a mucoadhesive hydrogel (i.e. in situ gelation process). In another embodiment, the ocular delivery system of the invention is under hydrated form, i.e. the preactivated thiomer matrix is hydrated under the form of a hydrogel.

The solid or semisolid ocular delivery system of the invention may be an ocular insert or an ocular film.

In one embodiment, the solid or semisolid ocular delivery system of the invention is an ocular insert, i.e. a solid or semisolid consistency tridimensional device designed to be placed into the conjunctival cul-de-sac or at the conjunctival surface, whose size and shape are especially designed for ophthalmic application. In one embodiment, the ocular insert is under dry form. In another embodiment, the ocular insert is hydrated and is under the form of a hydrogel pellet.

In one embodiment, the ocular delivery system of the invention an electrospun ocular insert. Preferably the ocular insert is solely formed from the preactivated thiomer matrix optionally comprising an active substance, i.e. the ocular insert does not comprise any additional layers or materials.

Ocular inserts can be obtained by direct compression of preactivated thiomers, which can be under lyophilized form. When comprising an active substance, ocular inserts can be obtained by direct compression of a mixture of active substance dispersed in preactivated thiomer.

The ocular insert can be of any shape and size, provided that it is suitable for ocular placement, and preferably is in shape of a rod, strip, thread, doughnut, disc, oval or quarter moon. In one embodiment, the ocular insert has one side convex and one side concave.

The ocular insert does not display any angle on its surface and presents a smooth surface that does not cause irritation to the eye nor to the eyelids. Preferably the cross section of the ocular insert is circular, square or rectangular. Preferably the insert is sized and shaped to readily fit into the eye, or a part thereof. In one embodiment, the ocular insert has a thickness ranging from 0.1 mm to 5 mm, preferably from 0.5 mm to 2 mm, more preferably from 0.5 mm to 1.5 mm. In one embodiment, the ocular insert has a length ranging from 1 mm to 10 mm, preferably from 2 mm to 5 mm. In one embodiment, the ocular insert has a width ranging from 1 mm to 10 mm, preferably from 2 mm to 5 mm.

In another embodiment, the solid or semisolid ocular delivery system of the invention is an ocular film, i.e. a solid or semisolid consistency bidimensional film designed to be placed into the conjunctival cul-de-sac or at the conjunctival surface, whose size and shape are especially designed for ophthalmic application. In one embodiment, the ocular film is under dry form. In another embodiment, the ocular film is under hydrated form.

The ocular film can be square shaped, circular, ellipsoid or any other suitable shape. Preferably, the ocular film has a thickness ranging from 0.01 μm to 1000 μm, preferably 0.5 μm to 500 μm. In case the ocular film is a circular film, it may have a diameter ranging from 2 mm to 20 mm, preferably from 5 mm to 10 mm. The ocular film can also have a curvature for suitable placement on the surface of the eye.

Ocular films can be obtained by solvent evaporation of preactivated thiolated polymer solutions, optionally comprising an active substance. Alternatively, ocular films can also be obtained by printing technologies, such as for example inkjet printing.

The present invention further relates to a kit comprising the solid or semisolid ocular delivery system of the invention. The kit may comprise instructions for use in the treatment and/or prevention of ocular diseases or ocular conditions. The kit may also comprise an applicator, preferably a sterile applicator.

### EXAMPLES

The present invention is further illustrated by the following examples.

### Example 1: Ocular Insert

An ocular insert comprising (i) a preactivated thiomer matrix of poly(acrylic acid)-cysteine-2-mercaptonicotinic acid and (ii) bimatoprost as active substance, was prepared by direct compression of co-lyophilized active substance and preactivated thiomer.

TABLE 1
Composition of the bimatoprost preactivated
thiolated PAA ocular insert

| Component | Type | Amount |
|---|---|---|
| poly(acrylic acid)-cysteine-2- | Preactivated thiomer | 2085 mg |
| mercaptonicotinic acid | | |
| (PAA-cys-2MNA) | | |
| bimatoprost | Active substance | 15 mg |

The preactivated thiomer poly(acrylic acid)-cysteine-2-mercaptonicotinic acid was synthesized as previously described (Iqbal J. et al., Biomaterials, 2012, 33, 1528-1535).

Bimatoprost was dispersed in an aqueous solution of preactivated thiomer and the dispersion was lyophilized Inserts of 1.5 mg, 2 mm in diameter, were prepared by tableting the lyophilized powder by direct compression.

The resulting ocular insert presents suitable properties for ocular use (size, shape, stability, cohesion), good mucoadhesive properties and is well tolerated. Bimatoprost is released from the ocular insert with a suitable release profile.

### Example 2: Ocular Film

An ocular film comprising (i) a preactivated thiomer matrix of chitosan-cysteine-6-mercaptonicotinic acid and (ii) dexamethasone phosphate sodium as active substance, was prepared by solvent evaporation.

TABLE 2
Composition of the dexamethasone preactivated
thiolated chitosan ocular film

| Component | Type | Amount (% w/w) |
|---|---|---|
| Chitosan-cysteine-6- | Preactivated thiomer | 99.7 |
| mercaptonicotinic acid | | |
| Dexamethasone phosphate | Active substance | 0.1 |
| sodium | | |
| Glycerol | Plasticizer | 0.2 |

Amount in weight percentage of the total weight of the film (% w/w).

The preactivated thiomer chitosan cysteine-6-mercaptonicotinic acid was synthesized as previously reported.

The film was obtained by solvent casting. Dexamethasone phosphate sodium, the preactivated thiomer and glycerol are dissolved in a solvent system of choice, casted and dried in hot air oven (40° C.-50° C.) and then cut in unit dose.

The resulting ocular film presents suitable properties for ocular use (size, shape, stability, cohesion), good mucoadhesive properties and is well tolerated. Dexamethasone is released from the ocular film with a suitable release profile.

### Example 3: In Vitro Characterization of Ocular Inserts

The ocular inserts of the invention are characterized with regard to their bioadhesive properties and swelling upon hydration.

Bioadhesion Assay:

Purpose: This assay aims at determining the mucoadhesive properties of the inserts of the invention by measuring the duration of bioadhesion of the inserts on animal mucosa under continuous flow of biological fluid.

Method: The insert is deposited on a section of animal mucosa and 10 μl of biological fluid is applied to allow for adhesion of the insert to the mucosa. The section of animal mucosa is then placed on a supporting glass platform which makes a 45° angle with the horizontal. Biological fluid is continuously flowing on the glass platform thanks to a reservoir and a peristaltic pump placed on the upper side of the glass platform. The biological fluid is flowing on the insert adhering to the mucosa and the elution fluid is collected on the lower side of the glass platform. The time at which the insert is dissolved or detached from the animal mucosa is determined. Thus, the duration of the adhesion of the inserts on the animal mucosa is measured.

In addition, the elution fluid collected at predetermined time points is assayed to quantify the drug released from the insert overtime.

Swelling and Hydration Assay:

Purpose: This assay aims at determining the hydratation properties and swelling behavior of the inserts of the invention by measuring their ability to uptake water and form a gel of defined dimensions.

Method: The method to determine water uptake and swelling behavior was adapted from the method previously described by Hornof et al. (Hornof M. et al., Journal of Controlled Release, 2003, 89, 419-428). Water uptake was determined gravimetrically. Inserts were hydrated with a defined volume of simulated lacrimal fluid in a closed container to prevent evaporation during the assay and incubated at 32° C., the eye surface temperature.

The weight of the inserts was determined at different time points to assess the speed of water uptake. The size of the inserts was measured macroscopically to determine the swelling propension of the inserts.

### Example 4: In Vivo Evaluation of Ocular Inserts

Purpose: The ocular inserts of the invention are tested in vivo in terms of adhesion to the ocular surface and harmlessness (absence of deterioration of ocular surface, no conjunctival/corneal inflammation or conjunctival/corneal infection, no eye pain).

Method: The assay was conducted on rats. The animals are kept in controlled conditions. Before any experimental procedure, a thorough examination of the ocular surface is performed on all rats (slit lamp, fluorescein test, conical mechanical sensitivity, in vivo confocal microscopy) to verify the absence of inflammation or infection, or even damage or injury to the ocular surface in non-implanted animals.

The animal is anesthetized by gas anesthesia in order to unilaterally place the tested insert in the lower conjunctival sac of the rat eye. The insert is left in place for seven days on the animal's eye in order to follow its fate. At the end of the application of the insert, the gas anesthesia is stopped allowing a rapid awakening of the animal. The spontaneous behavior of the animal observed. Then, a clinical evaluation of the integrity of the ocular surface (slit lamp, fluorescein test, presence of the insert in the conjunctival sac) and of the conical mechanical sensitivity (von Frey test), spontaneous pain (index of closure of the palpebral cleft) is performed regularly during the following seven days on vigilant animals. On day seven, a thorough examination of the ocular surface is performed by in vivo confocal microscopy, under general anesthesia of the animal. The implants are removed and quickly frozen in order to carry out a bacteriological study.

1.-12. (canceled)

13. A mucoadhesive solid or semisolid ocular delivery system comprising a preactivated thiomer matrix, wherein the matrix comprises at least one preactivated thiomer selected from polymeric compounds bearing 2-mercaptonicotinic acid, 6-mercaptonicotinic acid, 2-mercaptonicotinamide, 2-mercaptoisonicotinamide, 6-mercaptonicotinamide, 6-mercaptoisonicotinamide, 6,6′-dithionicotinamide or 6-mercaptopyridoxine side chains covalently bonded through disulfide bonds to a thiolated polymer backbone.

14. The mucoadhesive solid or semisolid ocular delivery system according to claim 13, which is an ocular insert or an ocular film.

15. The mucoadhesive solid or semisolid ocular delivery system according to claim 13, wherein the polymer backbone is selected from a (crosslinked) homo- or co-polymer consisting of (meth)acrylic acid, (meth)acrylic acid esters, (meth)acrylamides, vinylpyrrolidone, vinylalcohol, vinylimidazole, vinylcaprolactam, divinyl glycol, polycarbophil, carbomer, allylamine, (trimethylated) chitosans, hyaluronic acid, pectins, alginates, (crosslinked) polyallylamines, polylysine, polyornithine, polyaminoamides, methylcellulose, ethylcellulose, hydroxyethylcellulose, hydroxypropylcellulose, hydroxypropylmethylcellulose, and sodium carboxymethylcellulose, optionally exhibiting free thiol groups as side chains.

16. The mucoadhesive solid or semisolid ocular delivery system according to claim 13, wherein the side chains of the polymeric compound are selected from: S-(2- or 6-mercapto(iso)nicotinamide)- or S-(6-mercaptopyridoxine)-cysteine-disulfides, S-(2- or 6-mercapto(iso)nicotinamide)- or S-(6-mercaptopyridoxine)-homocysteine-disulfides, S-(2- or 6-mercapto(iso)nicotinamide)- or S-(6-mercaptopyridoxine)-cysteamine-disulfides, S-(2- or 6-mercapto(iso)nicotinamide)- or S-(6-mercaptopyridoxine)-N-acetylcysteine-disulfides, S-(2- or 6-mercapto(iso)nicotinamide)- or S-(6-mercaptopyridoxine)-thioglycolic acid-disulfides, S-(2- or 6-mercapto(iso)nicotinamide)- or S-(6-mercaptopyridoxine) thiopropionic acid-disulfides, S-(2- or 6-mercapto(iso)nicotinamide)- or S-(6-mercaptopyridoxine)-4-thiobutanoic acid-disulfides, S-(2- or 6-mercapto(iso)nicotinamide)- or S-(6-mercaptopyridoxine)-mercaptobenzoic acid-disulfides, S-(2- or 6-mercapto(iso)nicotinamide)- or S-(6-mercaptopyridoxine)-mercaptonicotinic acid-disulfides, S-(2- or 6-mercapto(iso)nicotinamide)- or S-(6-mercaptopyridoxine)-glutathione-disulfides, S-(2- or 6-mercapto(iso)nicotinamide)- or S-(6-mercaptopyridoxine)-thioethylamidine-disulfides, S-(2- or 6-mercapto(iso)nicotinamide)- or S-(6-mercaptopyridoxine)-4-thiobutylamidine-disulfides, and S-(2- or 6-mercapto(iso)nicotinamide)- or S-(6-mercaptopyridoxine)-mercaptoaniline-disulfides; said side chains being attached to the polymer backbone via amide, amidine or ester bonds.

17. The mucoadhesive solid or semisolid ocular delivery system according to claim 13, wherein the preactivated thiomer forming the matrix is under the form of nanofibers.

18. The mucoadhesive solid or semisolid ocular delivery system according to claim 17, wherein nanofibers are obtained by electrospinning.

19. The mucoadhesive solid or semisolid ocular delivery system according to claim 13, further comprising one or more pharmaceutically active substance.

20. The mucoadhesive solid or semisolid ocular delivery system according to claim 19, wherein the active substance is a pharmaceutically active substance selected from IOP lowering agents; anti-inflammatories; anti-infectives; anti-allergic agents; and dry eye treatment agents.

21. The mucoadhesive solid or semisolid ocular delivery system according to claim 20, wherein IOP lowering agents are selected from prostaglandin analogs, cholinomimetics, beta blockers, alpha adrenergic agonists, carbonic anhydrase inhibitors, Rho kinase inhibitors, NO donor agents and combinations thereof.

22. The mucoadhesive solid or semisolid ocular delivery system according to claim 20, wherein anti-inflammatories are selected from corticosteroid anti-inflammatory drugs and nonsteroidal anti-inflammatory drugs.

23. The mucoadhesive solid or semisolid ocular delivery system according to claim 20, wherein anti-infectives are selected from macrolides, aminosides, rifamycin, antiviral drugs and antifungal medication.

24. The mucoadhesive solid or semisolid ocular delivery system according to claim 20, wherein anti-allergic agents are selected from H1-antihistamines and mast cell stabilizers.

25. The mucoadhesive solid or semisolid ocular delivery system according to claim 13, further comprising one or more pharmaceutically acceptable excipient selected from: thickening agents, gelling agents, plasticizers, solubilization agents, stabilizing agents, permeation enhancers, diluents, binding agents, disintegrants, glidants, channeling agents and buffering agents.

26. The mucoadhesive solid or semisolid ocular delivery system according to claim 25, wherein thickening and gelling agents are selected from high molecular weight crosslinked polyacrylic acid polymers, polyvinyl alcohol, polyvinylpyrrolidone, cellulose derivatives, polyethylene glycol, and hyaluronic acid; plasticizer is glycerol; solubilization and stabilizing agents are selected from cyclodextrins; the permeation enhancer is glutathione in its reduced form; the diluents are selected from sugar alcohols; the binding agents are selected from saccharides and their derivatives, disaccharides, polysaccharides and their derivatives, cellulose, modified cellulose, sugar alcohols and synthetic polymers; disintegrants are selected from crosslinked polymers and modified starch; glidants are selected from magnesium stearate, dibasic calcium phosphate, starch, microcrystalline cellulose and colloidal silicon dioxide; and channeling agents are selected from sodium chloride and polyethylene glycol.

27. The mucoadhesive solid or semisolid ocular delivery system according to claim 13, comprising one or more pharmaceutically acceptable excipient selected from high molecular weight crosslinked polyacrylic acid polymers, polyvinyl alcohol, polyvinylpyrrolidone, cellulose, microcrystalline cellulose, hydroxypropyl cellulose, hydroxypropyl methylcellulose, carboxymethyl cellulose, polyethylene glycol, hyaluronic acid, glycerol, cyclodextrins, glutathione in its reduced form, sorbitol, xylitol, mannitol, saccharides and their derivatives, sucrose, lactose, polysaccharides and their derivatives, starches, sodium starch glycolate, magnesium stearate, dibasic calcium phosphate, colloidal silicon dioxide and sodium chloride.

28. A method for treating and/or preventing an ocular disease or ocular condition in a patient, comprising administering to target site of the eye of the patient in need thereof a mucoadhesive solid or semisolid ocular delivery system according to claim 13.

29. The method according to claim 28, wherein the ocular disease or ocular condition is selected from open angle glaucoma, macular edema, uveitis, dry eye disease, conjunctivitis, keratitis, blepharitis, endophthalmitis, trachoma, post-surgical and seasonal allergies.

30. A method for the manufacturing of a mucoadhesive solid or semisolid ocular delivery system, comprising forming the mucoadhesive solid or semisolid ocular delivery system from a preactivated thiomer, wherein the preactivated thiomer is selected from polymeric compounds bearing 2-mercaptonicotinic acid, 6-mercaptonicotinic acid, 2-mercaptonicotinamide, 2-mercaptoisonicotinamide, 6-mercaptonicotinamide, 6-mercaptoisonicotinamide, 6,6′-dithionicotinamide or 6-mercaptopyridoxine side chains covalently bonded through disulfide bonds to a thiolated polymer backbone.

31. The method according to claim 12, wherein the mucoadhesive solid or semisolid ocular delivery system is an ocular insert or an ocular film.
